(19) United States
(12) Patent Application Publication
(10) Pub. No.: US 2010/0160777 A1
(43) Pub. Date: Jun. 24, 2010

(54) REVERSE DEPLOYMENT DEVICE

(76) Inventors: Terry D. Hardin, Irvine, CA (US); Michael E. Miller, Trafalgar, IN (US); Aaron P. Barr, Fishers, IN (US); Michael Hoffa, Brownsburg, IN (US)

Correspondence Address: Hologic Cytyc-Suros Division;c/o Rader, Fishman & Grauer, PLLC, 39533 Woodward Avenue, Suite 140, Bloomfield, MI, 48304, US

(21) Appl. No.: 12/341,601

(22) Filed: Dec. 22, 2008

(51) Int. Cl. A61B 6/00

(52) U.S. Cl. 600/431

(57) ABSTRACT

A site marker deployment device is disclosed. The site marker deployment device comprises an inner member and a deployment cannula. The inner member has a distal end. The deployment cannula has an open distal end. The deployment cannula is slidably disposed over the inner member and a site marker that is disposed within the deployment cannula adjacent the inner member at the distal end of the depolyment cannula. The inner member is configured to be held generally stationary relative to a target location as deployment cannula is selectively moved between a pre-deployment configuration wherein the deployment cannula is positioned over at least a portion of the site marker and a deployed configuration wherein the deployment cannula is retracted such that the site marker is released from the deployment cannula.

### TECHNICAL FIELD

The present disclosure relates generally to deployment mechanisms for deploying site markers in connection with biopsy procedures.

### BACKGROUND

In the diagnosis and treatment of cancer, it is often necessary to perform a biopsy to remove tissue samples from a suspicious mass. The suspicious mass is typically discovered during a preliminary examination involving visual examination, palpation, X-ray, magnetic resonance imaging (MRI), ultrasound imaging or other detection means.

When a suspicious mass is detected, a sample is talcen by biopsy, and then tested to determine whether the mass is malignant or benign. This biopsy procedure can be performed by an open surgical technique, or through the use of a specialized biopsy instrument such as stereotactic needle biopsy. To minimize surgical intrusion, a small specialized instrument such as a biopsy needle is inserted in the breast while the position of the needle is monitored using fluoroscopy, ultrasonic imaging, X-rays, MRI or other suitable imaging techniques.

Regardless of the method or instrument used to perform the biopsy, subsequent examination of the surgical site may be necessary, either in a follow up examination or for treatment of a cancerous lesion. In connection with breast biopsy procedures for example, treatment often includes a mastectomy, lumpectomy, radiation therapy, or chemotherapy procedure that requires the surgeon or radiologist to direct surgical or radiation treatment to the precise location of the lesion. Because this treatment might extend over days or weeks after the biopsy procedure, and the original features of the tissue may have been removed or altered by the biopsy, it is desirable to insert a site marker into the surgical cavity to serve as a landmark for future identification of the location of the lesion.

Commonly assigned application U.S. patent application Ser. No. 11,242,334 discloses markers that use expandable filament portions to ‘hold’ a site marker in place within a biopsy cavity. That is, a site marker may include a bio-absorbable filament portion, such as a suture, with a marker attached thereto, where the marker is visible under multiple modalities and the suture will inhibit migration of the marker within the biopsy cavity. The filament portions of these structures typically define a site marker diameter that is greater than the outer diameter of the cannula. To insert a site marker within a biopsy site, the site marker is compressed (at least partially elastically deformed) to a dimension that will permit the site marker to be interposed within the cannula, the site marker is interposed within an opening of the cannula, the site marker and cannula are sterilized, the cannula is inserted within the biopsy canal such that the opening is within the biopsy site, and the marker is deployed by projecting it into the biopsy site. Once deployed, the site marker will expand as the filament portions exit the cannula in reaction to the elastic deformation. The site marker will expand until the elastic deformation is eliminated or portions of the site marker interfere with the inside portions of the biopsy cavity.

Precise placement of site markers is important for subsequent evaluation of the biopsy area. Current methods of marker deployment generally include locating the outer cannula of the biopsy instrument in the area of the lesion, then depressing a plunger or pushrod within the cannula to force the marker into a biopsy cavity. When the plunger or pushrod is depressed, however, the site marker may not be placed exactly where intended. For instance, if the cannula is placed at the center of a biopsy cavity, the marker may move from the center of the cavity to one side, making the marker more difficult to subsequently locate. Furthermore, varying amounts of pressure on the pushrod may cause the site marker to extend further into the cavity than desired.

Accordingly, there is a need for a site marker deployment method which allows more precise placement of a site marker within a biopsy cavity.

### SUMMARY

A site marker deployment device is disclosed. The site marker deployment device comprises an inner member and a deployment cannula. The inner member has a distal end. The deployment cannula has an open distal end. The deployment cannula is slidably disposed over the inner member and a site marker that is disposed within the deployment cannula adjacent the inner member at the distal end of the depolyment cannula. The inner member is configured to be held generally stationary relative to a target location as deployment cannula is selectively moved between a pre-deployment configuration wherein the deployment cannula is positioned over at least a portion of the site marker and a deployed configuration wherein the deployment cannula is retracted such that the site marker is free from the deployment cannula.

### BRIEF DESCRIPTION OF THE DRAWINGS

FIG. 1 is a perspective view of a biopsy site in a human breast showing breast tissue in section and a site marker being implanted in a biopsy cavity using a site marker delivery system according to an embodiment.

FIG. 2 is a partially sectioned side view of a site marker and deployment device in a pre-deployment configuration according to an embodiment.

FIG. 3 is another partially sectioned side view of the site marker and deployment device of FIG. 2 in a pre-deployment configuration according to an embodiment.

FIG. 4 is another partially sectioned side view of the site marker and deployment device of FIG. 2 in a deployment configuration according to an embodiment.

FIG. 4a is an enlarged view of a distal end of a restraining member attached to a site marker.

FIG. 5 is a partially sectioned side view of a site marker and deployment device in a pre-deployment configuration according to an embodiment.

FIG. 6 is a partially sectioned side view of the site marker and deployment device in a deployment configuration.

FIG. 7 is a side view of a site marker disposed within a cannula in a pre-deployment configuration in accordance with an embodiment.

FIG. 8 is a side view of the marker of FIG. 7 in a deployed configuration.

FIG. 9 is a side view of a site marker disposed within a cannula in a pre-deployment configuration in accordance with an embodiment.

FIG. 10A is a side view of a site marker disposed within a cannula in a pre-deployment configuration in accordance with an embodiment.

FIG. 10B is a side view of the site marker of FIG. 10A in a deployed configuration.

FIG. 11A is a side view of a site marker being loaded into a cannula through a marker loading tool.

FIG. 11B is a side view of the site marker of FIG. 11A in a pre-deployment configuration after being loaded into the cannula through the marker loading tool.

### DETAILED DESCRIPTION

Referring now to the drawings, illustrative embodiments are shown in detail. Although the drawings represent some embodiments, the drawings are not necessarily to scale and certain features may be exaggerated, removed, or partially sectioned to better illustrate and explain the present disclosure. Further, the embodiments set forth herein are not intended to be exhaustive or otherwise limit or restrict the claims to the precise forms and configurations shown in the drawings and disclosed in the following detailed description. While the disclosure is described in connection with reference to a human breast, it is understood that the present disclosure may be employed with other areas of the body in which a site marker may be utilized.

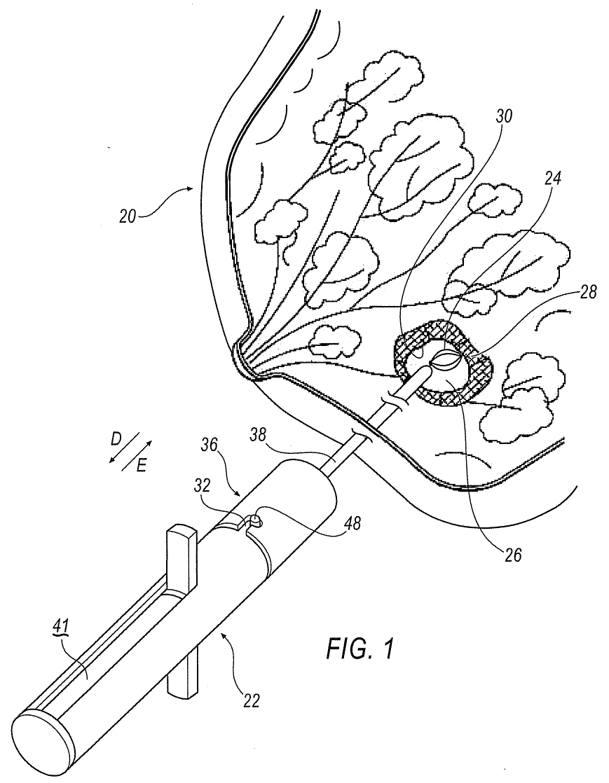

FIG. 1 illustrates a perspective view of a human breast, or tissue, 20 and a site marker deployment system 22. As illustrated, the tissue 20 is being implanted with a site marker 24 at a biopsy site 26. In the embodiment illustrated, the biopsy site 26 is created in a lesion 28 from which a tissue sample (not shown) has been removed, resulting in a biopsy cavity 30. One or more site markers 24 may be implanted in the biopsy cavity 30 using the system 22. In one embodiment, the site marker delivery system 22 is slidably advanced through an outer cannula of a biopsy device (not shown), which avoids the need to withdraw the biopsy device and thereafter insert the marker delivery system 22. An exemplary biopsy device through which the site marker delivery system of the present invention may be advanced is illustrated in commonly owned U.S. Pat. Nos. 6,638,235 and 6,758,824, the contents of which are incorporated in its entirety. Delivering the site marker 24 in the biopsy cavity 30 without withdrawing the biopsy device reduces the amount of tissue damage and enables more accurate placement of the site marker 24.

Alternatively, the site marker delivery system of the present invention may be advanced through an introducer assembly that includes an outer introducer cannula 38. An exemplary introducer assembly is illustrated in commonly owned U.S. Pat. No. 7,347,829, the contents of which are incorporated by reference in its entirety. However, the system 22 illustrated in FIG. 1 is exemplary only.

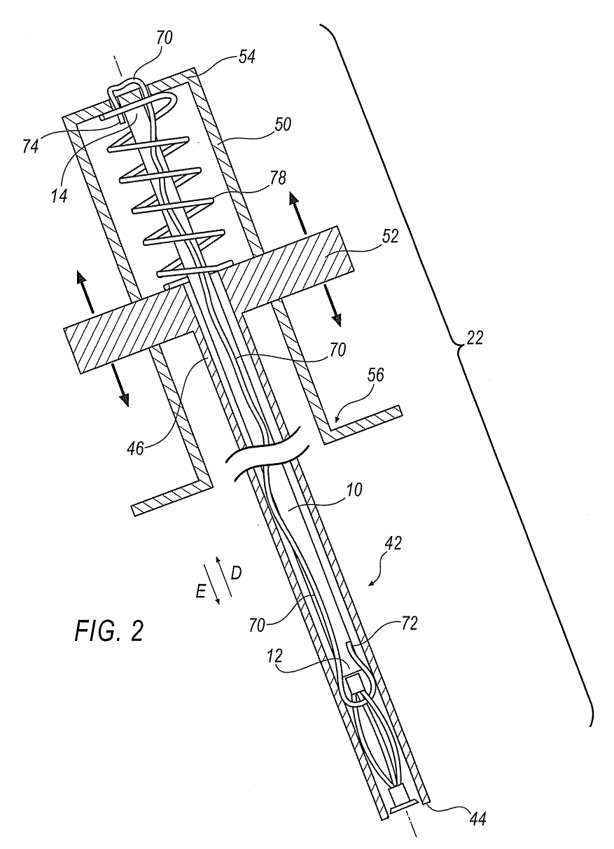

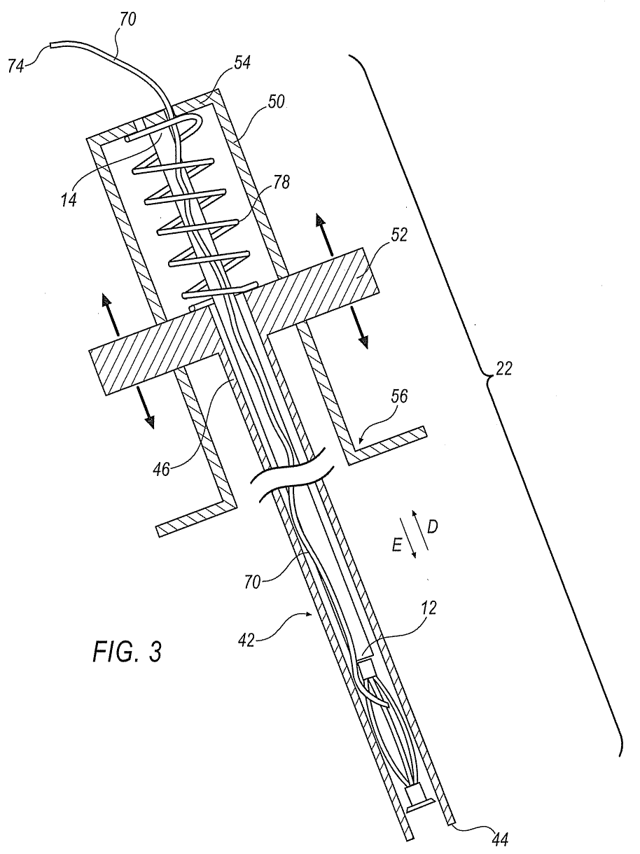

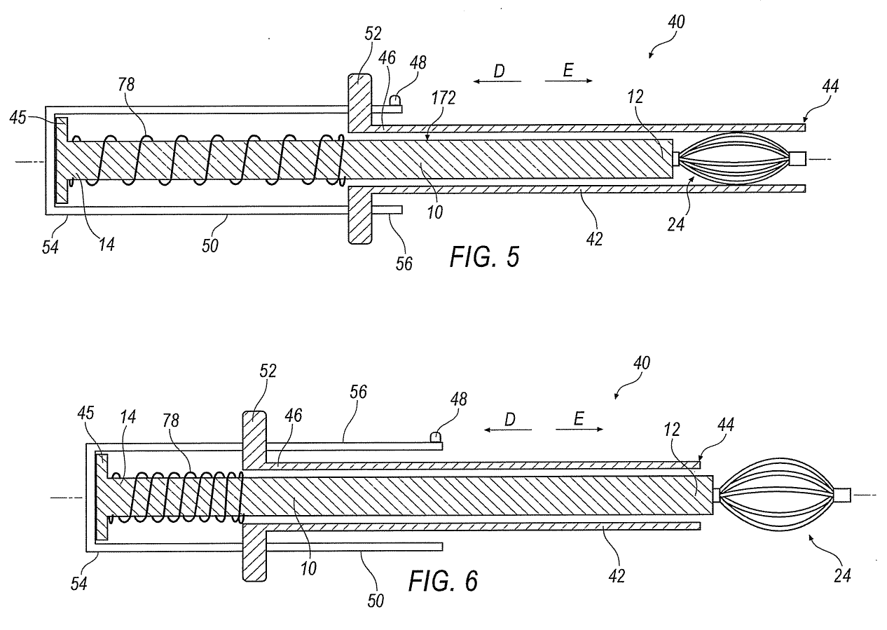

FIGS. 2-4 illustrate an embodiment of a deployment device 40 that may be used in system 22. The illustrated device 40 includes an inner member 10 defined by a distal end 12 and a proximal end 14. In one embodiment, proximal end 14 is fixedly connected to a body 50 having a proximal end 54 and a distal end 56. The deployment device 40 further includes a deployment cannula portion 42 disposed around at least a portion of the inner member 10 when deployment device 40 is in a pre-deployment configuration, as shown in FIGS. 2-3. One or more handle members 52 may be provided that are operatively connected to the deployment cannula 42. Handle members 52 are configured so as to be slidably disposed within a channel 41 (shown in FIG. 1) formed in body 50. A spring member 78 is disposed between handle members 52 and proximal end 54 of body 50, around proximal end of inner member 10. In one embodiment, proximal end 54 of body 50 may be configured with a mounting flange (shown in FIGS. 5 and 6) to assist in retaining spring member 78. Spring member 78 may interact with the deployment cannula 42 to prevent premature retraction of the deployment cannula 42, as will be explained in further detail below.

Site marker 24 is disposed within deployment cannula 42, adjacent distal end 12 of inner member 10. The site marker 24 may be at least partially enclosed within a distal end 44 of deployment cannula 42, as shown in FIG. 2.

The deployment device 40 may further include a retaining member 70, such as a trigger wire, disposed therein. The trigger wire 70 extends from a distal wire end 72 near a distal end 12 of the inner member 10 toward a proximal end 54 of the body 50. The illustrated trigger wire 70 is configured to retain a site marker 24 relative to the inner member 10, such as after sterilization and prior to enclosure within the deployment carnula 42. The site marker 24 may be held relative to the inner member 1 0 as deployment cannula 42 is extended axially relative to inner member 10, in the direction of the distal end 12 of the inner member 10, until the deployment cannula 42 at least partially encloses the site marker 24. The trigger wire 70 may further retain the site marker 24 relative to the inner member 10 while the deployment cannula 42 is inserted into the biopsy cavity 30 in the tissue 20 to a target location.

Referring to FIG. 3, the trigger wire 70 may be subsequently disengaged from the site marker 24, such as by moving the trigger wire 70 in the direction of the arrow D. Alternatively, the trigger wire 70 may be disengaged from the site marker 24 prior to insertion of the deployment cannula into the tissue 20.

FIG. 3 illustrates trigger wire 70 being disengaged from site marker 24, prior to deployment of the site marker 24. The proximal end 74 of trigger wire 70 is retracted from a proximal end 54 of the body 50 and pulled in the direction of the arrow D. As the trigger wire 70 is removed, the distal end 72 of the trigger wire 70 straightens, releasing site marker 24. The trigger wire 70 may be completely removed from the device 40, or may be only removed to a sufficient extent that site marker 24 is disengaged therefrom.

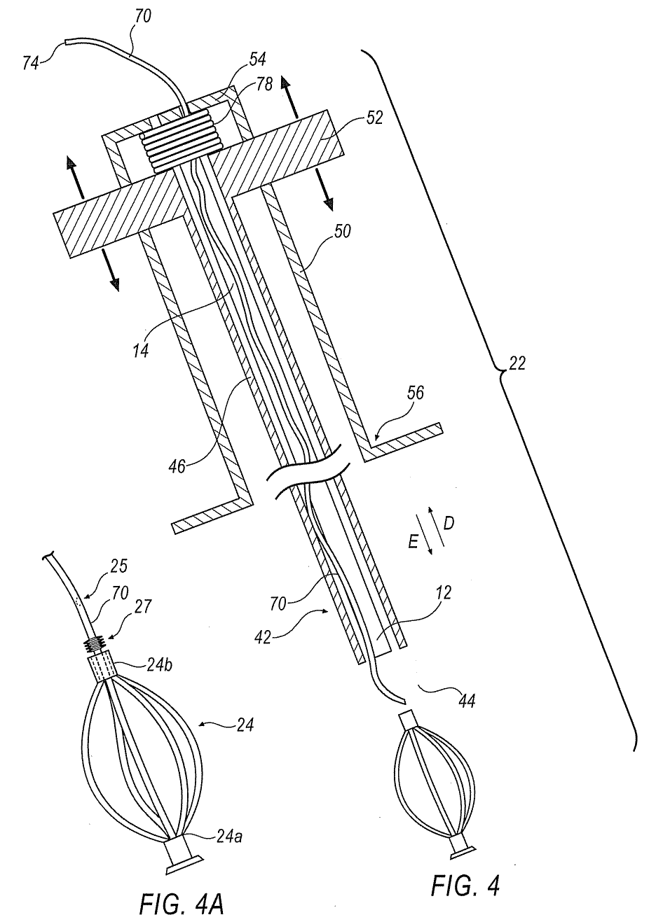

FIG. 4 illustrates the deployment of the site marker 24, after the trigger wire 70 has been detached from the site marker 24. When the deployment cannula 42 is placed within the biopsy cavity 30 at a target location, the deployment cannula 42 may be moved in the direction of the arrow D, such as by pulling back on the one or more handle members 52, to release the site marker 24. In the embodiment illustrated, a distal end 12 of inner member 10 is configured with a length that may extend to the distal end 44 of the deployment cannula 42, or may extend past the distal end 44 of the deployment cannula 42, such that site marker 24 is forced outward of distal end 44 of deployment cannula. In other words, site marker 24 is released within cavity 30 upon retraction of the cannula 42.

In another exemplary embodiment, after deployment cannula 42 is inserted into biopsy cavity 30, deployment cannula 42 may be retracted by moving handle member in direction D, prior to trigger wire 70 being detached from site marker 24. Thus, in this exemplary embodiment, trigger wire 70 serves to hold site marker 24 against distal end 12 of inner member 10 within biopsy cavity 30, to insure proper placement of site marker 24 within biopsy cavity 30 prior to releasing site marker 24 from deployment device 40.

In yet another alternative embodiment (referring to FIG. 4A) trigger wire 70 may be configured with site marker 24 so as to serve as a deployment line that is attached to a distal end 24a of site marker 24 and extends through site marker 24 and out through a proximal end 24b of site marker 24. When deployment device 40 is inserted into biopsy cavity 30, and deployment cannula 42 is retracted in direction D, trigger wire 70 is then pulled in direction D. As inner member 10 keeps site marker 24 prevents site marker 24 from moving in direction D, trigger wire 70 will cause distal end 24a of site marker 24 to move toward proximal end 24b of site marker 24, thereby manually expanding site marker 24 from a pre-deployment configuration (such as shown in FIG. 3), to a deployment configuration (such as shown in FIG. 4). To insure that site marker 24 remains in the deployment configuration, a portion of trigger wire 70 may be configured with deformable retaining members 27 that bias into a retaining configuration (shown in FIG. 4A), once passed through proximal end 24b of site marker 24. Further, trigger wire 70 may also be provided with a weakened area 25 that will break apart after trigger wire 70 has been subjected to a predetermined force so as to release site marker 24 into biopsy cavity.

To ensure proper placement of the site marker 24 within the biopsy cavity 30 when the device 40 is placed in the target location, the site marker 24 is adjacent to the inner member 10 within the deployment cannula 42. The inner member 10 is held stationary relative to the target location by, for example, a tab 48 that is fixedly attached to body 50, as seen in FIGS. 1 and 5-6. More specifically, tab 48 may be slidably received within a notch 32 formed on a portion of a introducer member 36 that includes outer introducer cannula 38. Alternatively, tab 48 may be slidably received a notch that is formed on a proximal end of a biopsy device.

When the inner member 10 is in place, the deployment cannula 42 is withdrawn from the site marker 24 by retracting the handle member 52 in the direction of the arrow D, toward the proximal end 14 of the inner member 10. As the deployment cannula 42 is withdrawn, the inner member 10 cooperates with the site marker 24 to keep the site marker 24 in place. As the deployment cannula 42 is withdrawn, any friction between the site marker 24 and the deployment cannula 42 is overcome by the interaction between the site marker 24 and the inner member 10. By retracting the deployment cannula 42, as opposed to extending an inner member 10 outward of the distal end 44 of the deployment cannula 42, the precision with which the site marker 24 is placed at a desired location within a biopsy cavity is increased.

In one exemplary embodiment, one or more portions of site marker deployment system 22 may be constructed out of a radiolucent material, which is not visible under certain imaging modalities. Examples of suitable radiolucent materials include plastic, ceramic or glass. By constructing one or more portions of site marker deployment system 22 out of such radiolucent materials, the user will be able to confirm placement of the site marker 24 (while still encapsulated within the site marker delivery system 22) immediately prior to placement, but without compromising the ability of the imaging system to see any other suspicious areas underneath or around the site marker delivery system 22 during the imaging sequence.

FIGS. 5 and 6 illustrate a deployment device 40 according to a further embodiment. The embodiment in FIGS. 5 and 6 includes an inner member 10 having a proximal end 14 operatively connected with a body 50, and a distal end 12. The body 50 may further include a tab 48 for selective interaction with a notch 32 formed within housing 36 of an introducer, to retain the position of the inner member 10 relative to a target area. A deployment cannula 42 is selectively slidably disposed around at least a portion of the inner member 10. A proximal end 46 of the deployment cannula 42 is operatively connected with a handle portion 52. The handle portion 52 extends through a channel 41 formed through an outer face of the body 50, extending generally from a distal end 56 of the body 50 to a proximal end 54. A distal end 44 of the deployment cannula 42 defines an aperture therein. A site marker 24 is elastically deformed and selectively disposed within at least a portion of the aperture defined within the distal end 44 of the deployment cannula 42. The elastic deformation of the site marker 24 causes the site marker 24 to exert a force against an inner face of the deployment cannula 42, thereby frictionally holding the site marker 24 within the cannula 42.

Once deployment cannula 42 is pulled back in direction D, the inner member 10 keeps site marker 24 in place until site marker 24 is positioned outwardly from distal end 44 of deployment cannula 42. Once positioned outside of deployment cannula 42, site marker is biased to expand outwardly, thereby preventing re-entry of site marker 24 into deployment cannula 42. In one embodiment, once site marker 24 expands to its deployed configuration (FIG. 6), deployment cannula 42 may be moved in a reverse direction E such that distal end 44 of deployment cannula 44 may contact site marker 24 to disengage site marker 24 from inner member 10. In such an embodiment, site marker 24 is adhered to inner member 10 with a low strength adhesive that is easily overcome by movement of the deployment cannula 44 into contact with site marker 24 such that site malker 24 may be released from the deployment device 40.

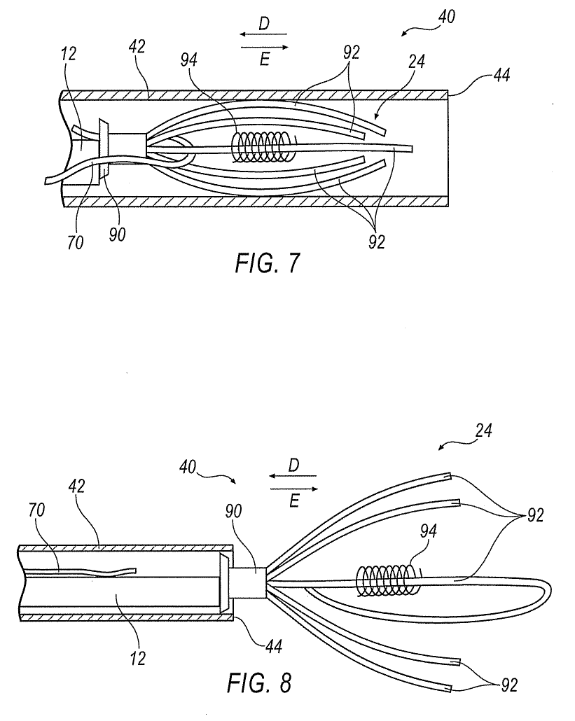

FIGS. 7 and 8 illustrate a portion of an embodiment of a deployment device 40, to further illustrate the manner of deployment of an embodiment of a site marker 24. The deployment device 40 includes a site marker 24 and a deployment cannula 42. To form the present embodiment of site marker 24, a wire or suture material 92 is attached to a marker end 90. The wire 92 may be constructed from any biocompatible material with suitable echogenic properties such as, but not limited to, titanium, stainless steel, or platinum. Alternatively, the wire 92 may be a bio-absorbable material with a marker element, or permanent marker, 94 attached to a portion thereof where the permanent marker 94 has suitable echogenic properties. In the embodiment illustrated, the permanent marker 94 includes an aperture with a wire 92 interposed therethrough.

The deployment device 40 also includes a trigger wire 70 and an inner member 10 interposed within the deployment cannula 42. The trigger wire 70 extends from a distal end 12 of the inner member 10 to a proximal end (not shown). The site marker 24 includes filament members 92 connected to the marker end 90. A central filament member 92 extending from the marker end 90 may be looped to retain the permanent marker 94.

The trigger wire 70 and the inner member 10 may be held rigid while the deployment cannula 42 is extended generally in the direction of arrow E as the site marker 24 is enclosed within the deployment cannula 42 toward the position of FIG. 7. Once enclosed to about the position of FIG. 7, the deployment device 40 may then be activated to deploy the site marker 24.

To deploy the site marker 24, the trigger wire 70 is pulled in the general direction of the arrow D as the deployment cannula 42 is held in a generally unmoved position relative to the inner member 10. Once the trigger wire 70 is pulled out of contact with the site marker 24, the deployment cannula 42 may be moved generally in the direction of the arrow D until the site marker 24 is no longer enclosed within the deployment cannula 42, thereby deploying the site marker 24.

Once released from the deployment device 40 and into the biopsy cavity 30, the site marker 24 automatically springs (due to the elastic deformation) into the deployed configuration having a size and shape generally defined by the biopsy cavity 30 such that the site marker 24 is easily visible under various imaging modalities.

In the embodiments illustrated, the permanent markers may be constructed of a material that is not absorbed by the body. Alternatively, the permanent markers may be a semi-permanent marker that bio-absorbs slower than the filament member. Because the movement of the permanent markers is restricted by the filament members prior to absorption thereof by the body, the permanent markers are restricted from migrating from within biopsy cavity. This insures that the permanent markers remain within the biopsy cavity to permit follow-up imaging of the biopsy site.

Since a site marker, such as the site marker 24, may be deployed with the aid of MRI, the user will visually detect when the site marker has been deployed and may confirm that the site marker has been successfully deployed in the desired location.

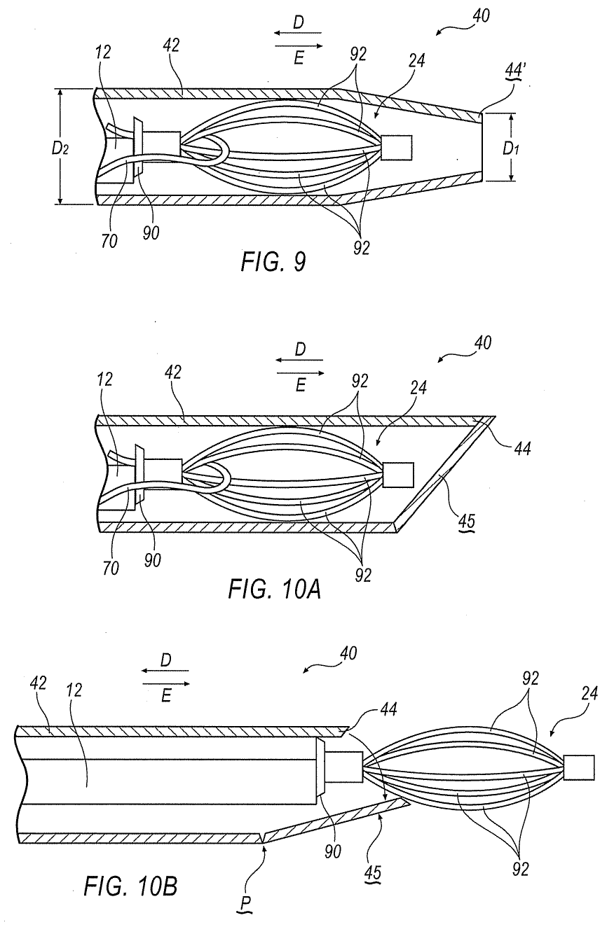

Referring now to FIG. 9, an alternative embodiment of a site marker deployment device 40 may be seen. Site marker deployment device 40 is similar to that shown in FIGS. 2-8, however, in the embodiment in FIG. 9, distal end 44′ of deployment cannula 42 is swaged such that the diameter D₁ of distal end 44′ is smaller than a diameter D₂ of a main portion of deployment cannula 42. The swaged distal end 44′ acts as a retaining mechanism to prevent accidental dislodgment of site marker 24 prior to intentional deployment.

Referring now to FIGS. 10A and 10B, another alternative embodiment of a site marker deployment device 40 may be seen. Site marker deployment device 40 is similar to the other embodiments previously discussed, except, however, that distal end 44 is formed with a hinged retaining member 45 that is biased in a closed position, as shown in FIG. 10A. When in the closed position, retaining member 45 serves to retain site marker 24 within deployment cannula 42 so as to prevent accidental dislodgment of site marker 24 prior to intentional deployment. However, turning now to FIG. 10B, when it is desired to deploy site marker 24, as deployment cannula 42 is moved in the deployment direction D, retaining member 45 is permitted to pivot about pivot point P so as to open retaining member 45 as shown in FIG. 10B. When in the open position, site marker 24 is permitted released from deployment cannula 42.

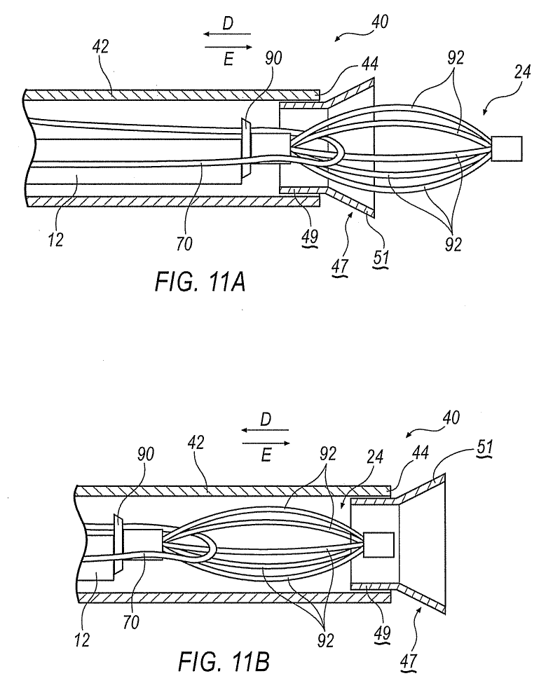

Turning now to FIGS. 11A-11B, a marker loading tool 47 is shown for use with a deployment device 40 will be discussed. Marker loading tool 47 includes a cannula end 49 and a flared end 51. Cannula end 49 is sized so as to fit within a distal end 44 of deployment cannula 42, with flared end 51 extending outwardly from distal end 44.

Once marker loading tool 47 is inserted into deployment cannula 42, trigger wire 70 is threaded through deployment cannula 42 so as to loop around a portion of site marker 24, which is positioned outwardly from distal end 44. Once looped around site marker 24, trigger wire 70 is then retracted in direction D so as to pull site marker 24 through marker loading tool 47. Because marker loading tool 47 includes an inwardly flared end 51, as positioned within deployment cannula 42, site marker 24 is compressed into a pre-deployment configuration (shown in FIG. 11B) as trigger wire 70 moves site marker 24 into deployment cannula 42 so as to position site marker 24 therein. Once positioned within deployment device 40, marker loading tool 47 may be removed from distal end 44 of deployment cannula 42. Marker loading tool 47 permits both the deployment device 40 and site markers 24 to be sterilized prior to loading site markers 24 within deployment device 40, thereby reducing the potential for excessive plastic deformation of site markers 24 due to the application of heat during the sterilization process.

Although the steps of deploying the site markers described herein are listed in a particular order, the steps may be performed in differing orders or combined such that one operation may perform multiple steps. Furthermore, a step or steps may be initiated before another step or steps are completed, or a step or steps may be initiated and completed after initiation and before completion of (during the performance of) other steps.

While the embodiments of site markers 24 are described as having multiple filament members, it is understood that one or more filament members may be adequate to retain the marker in the desired biopsy cavity or other location. In addition, while the present invention has been particularly shown and described with reference to the foregoing preferred embodiments, it should be understood by those skilled in the art that various alternatives to the embodiments of the invention described herein may be employed in practicing the invention without departing from the spirit and scope of the invention as defined in the following claims. It is intended that the following claims define the scope of the invention embodiments within the scope of these claims and their equivalents be covered thereby. This description of the invention should be understood to include all novel and non-obvious combinations of elements described herein, and claims may be presented in this or a later application to any novel and non-obvious combination of these elements. The foregoing embodiment is illustrative, and no single feature or element is essential to all possible combinations that may be claimed in this or a later application.

What is claimed is:

1. A site marker deployment device comprising:
an inner member having a distal end; and
a deployment cannula having an open distal end;
wherein the deployment cannula is slidably disposed over the inner member and a site marker is disposed within the deployment cannula adjacent the inner member at the distal end of the depolyment cannula; and
wherein the inner member is configured to be held generally stationary relative to a target location as deployment cannula is selectively moved between a pre-deployment configuration wherein the deployment cannula is positioned over at least a portion of the site marker and a deployed configuration wherein the deployment cannula is retracted such that the site marker is released from the deployment cannula.

2. The device of claim 1 wherein the deployment device includes a spring member that biases the deployment cannula to a pre-deployment configuration.

3. The device of claim 1 wherein a portion of the inner member extends outwardly from the open distal end of the deployment cannula when the deployment cannula is in the deployed configuration.

4. The device of claim 1 wherein the device is configured to retain the site marker generally stationary relative to a target location.

5. The device of claim 1 wherein the site marker includes a generally elongated first filament member, the first filament member selectively configurable between a retracted configuration, wherein the site marker is selectively interposed within the deployment cannula, and a deployed configuration, where the site marker cannot be interposed within the deployment cannula, and wherein a portion of the site marker is elastically deformed when the site marker is interposed within the deployment cannula.

6. The device of claim 1 further including a restraining member selectively disposed within the deployment cannula and selectively engaging the site marker.

7. The device of claim 6 wherein the restraining member is configured to selectively release the site marker when the deployment cannula is retracted in the deployed configuration.

8. The device of claim 1, further including a handle member that is operatively connected to the deployment cannula.

9. The device of claim 1, further including at least one tab member that cooperates with an external device to lock the position of the deployment device with respect to a target device.

10. The device of claim 1, further comprising a body portion to which the inner member is fixed.

11. The device of claim 10, wherein the deployment device is selectively slidable with respect to the body portion.

12. The device of claim 1, wherein the distal end of the deployment cannula is swaged so as to narrow from a first diameter to a second diameter.

13. The device of claim 1, wherein the distal end of the deployment cannula includes a selectively openable distal end that is closed off by a retaining member, wherein the retaining member may be selectively opened when the deployment cannula is retracted.

14. The device of claim 13, wherein the retaining member is hingedly connected to the deployment cannula.

15. The device of claim 1, further including a marker loading tool that is selectively received within the deployment cannula.

16. The device of claim 15, wherein the marker loading tool comprises a cannula end and an outwardly flared end, wherein the cannula end is sized to be received within the distal end of the deployment cannula.

17. The device of claim 1, wherein at least a portion of the site marker deployment device is constructed of a radiolucent material.

18. The device of claim 17, wherein the radiolucent material is one of plastic, ceramic and glass.

19. A method of deploying a site marker comprising:
providing a deployment device having a deployment cannula slidably disposed against an inner member, and having a site marker selectively disposed within the deployment cannula proximate to a distal end of the inner member;
retaining the site marker generally stationary relative to a target location; and
retracting the deployment cannula from the distal end of the inner member, thereby exposing the site marker and releasing the site marker from the deployment cannula.

20. The method of claim 19 further comprising placing a marker loading tool in the distal end of the deployment cannula and retracting the site marker through the marker loading tool into the deployment cannula so as to dispose the site marker within the deployment cannula proximate to a distal end of the inner member.

21. The method of claim 19 further comprising the steps of:
providing at least one restraining member operatively retaining the site marker within the deployment cannula prior to deployment; and
removing the restraining member from the site marker to permit deployment of the site marker.

22. The method of claim 19, further comprising opening a retaining member that is positioned at a distal end of the deployment device to permit deployment of the site marker.
